(19) United States
(12) Patent Application Publication
(10) Pub. No.: US 2011/0237452 A1
(43) Pub. Date: Sep. 29, 2011

(54) MARKER FOR DIAGNOSIS OF EXPOSURE TO ELECTROMAGNETIC RADIATION AND DIAGNOSTIC KIT COMPRISING THE SAME

(75) Inventors: Woong Yang Park, Seoul (KR); Ae Kyung Park, Seoul (KR); Chang Nim Im, Seoul (KR); Sook Ik Jeon, Daejeon (KR)

(73) Assignees: SNU R&DB FOUNDATION, Seoul (KR); ELECTRONICS AND TELECOMMUNICATIONS RESEARCH INSTITUTE, Daejeon (KR)

(21) Appl. No.: 13/033,763

(22) Filed: Feb. 24, 2011

(30) Foreign Application Priority Data
Feb. 25, 2010 (KR) 10-2010-0017364

(51) Int. Cl. C40B 30/04; C07H 21/04; C07K 16/00; C40B 40/08; C12Q 1/68; G01N 33/50; G01N 33/53

(52) U.S. Cl. 506/9; 536/24.33; 536/24.3; 530/387.1; 506/17; 435/6.12; 436/94; 435/7.92; 436/501

(57) ABSTRACT

Disclosed are a composition for the diagnosis of exposure to electromagnetic radiation, comprising an agent capable of measuring the expression level of the diagnostic marker, a diagnosis kit comprising the same, a method for detecting the diagnostic marker, and a method for the diagnosis of exposure to electromagnetic radiation. The diagnostic markers are very useful for monitoring and diagnosing exposure to electromagnetic fields, and can be used as instruments by which physiological mechanisms incurred upon electromagnetic radiation exposure are examined.

### CROSS REFERENCE TO RELATED APPLICATIONS

This application claims priority to Korean Patent Application No. 10-2010-0017364, filed: Feb. 25, 2010, which is hereby incorporated by reference in its entirety.

### BACKGROUND OF THE INVENTION

1. Field of the Invention

The present invention relates to a composition for the diagnosis of exposure to electromagnetic radiation, comprising an agent capable of measuring the expression level of the diagnostic marker, a diagnosis kit comprising the same, a method for detecting the diagnostic marker, and a method for the diagnosis of exposure to electromagnetic radiation.

2. Description of the Related Art

Modem people, whether in their workplaces or at home, are inevitably exposed to electromagnetic waves. Almost all the electronic and electric devices with which modern people live every day, including mobile phones, personal computers, television sets, electric shavers, etc. radiate electromagnetic waves. With the increasing controversy about the hazard of electromagnetic waves, the Ministry of Health and Welfare, Republic of Korea, announced “a warning report about exposure to electromagnetic radiation” in 1996, which describes the malfeasance of electromagnetic waves, recommending less exposure to electromagnetic waves.

When an electric current flows, an electric field occurs with the concomitant generation of a magnetic field around the flow of the electric current. The fields change periodically, producing waves, that is, electromagnetic waves. Electromagnetic waves exist wherever electric currents flow.

Radiofrequency (RF) radiation, a type of electromagnetic waves, finds various applications in daily life-related fields including TV broadcasting, mobile radio communication, computer networks, etc., and numerous other applications. Although the energy level of RF radiation is not high enough to break covalent bonds, it can induce molecular responses, leading to cell proliferation or cell death (Moulder, J. E. et al., (1999) Cell phones and cancer: what is the evidence for a connection? Radiat Res 151, 513-531). Radiofrequency radiation itself has not a direct influence on DNA and proteins, but may induce the alteration of intracellular signaling pathways through changes in membrane fluidity or ion distribution. Further, interactions between genes and RF radiation induces various physiological conditions to lower the threshold of physiological changes.

For example, the brain is especially the most important target tissue to study the biological effects of RF radiation in mobile phone users (Hardell, L et al., (1999) Use of cellular telephones and the risk of brain tumors: a case control study. Int J Oncology 15, 113-116). Several electrophysiological studies have reported the alteration of cognitive and physiological function of the brain upon exposure to mobile phone-frequency RF radiation. In sum, RF exposure can induce measurable changes in human brain electrical activity, particularly in the alpha frequency band (8-13 Hz) over posterior regions of the scalp. Moreover, rats exposed to RF radiation showed neuronal damage in the cortex, hippocampus, and basal ganglia. However, there are a number of points to consider regarding whether RF radiation can affect the human brain and its subsequent output in the form of cognition and behavior.

Gene expression profiling using microarray can give important information on characteristic changes in physiological and pathological conditions. For example, gene expression profiles of irradiated Jurkat cells showed p53-independent way of the NF-κB pathway (Park, W. Y. et al., (2002) Identification of radiation-specific responses from gene expression profile. Oncogene 21, 8521-8528).

Leading to the present invention, intensive and thorough research, conducted by screening genes, which had changed their expression levels since exposure to electromagnetic radiation, and picking out ones which showed greatest changes in expression level through the observation of gene expression patterns, resulted in the finding that the genes of interest can be targets useful for examining whether the subject was exposed to electromagnetic radiation.

### SUMMARY OF THE INVENTION

It is therefore an object of the present invention to provide a composition for diagnosis of exposure to electromagnetic radiation, comprising agents capable of measuring at an mRNA or protein level the expression level of the genes given in Table 6.

It is another object of the present invention to provide a kit for the diagnosis of exposure to electromagnetic radiation, comprising the composition.

It is a further object of the present invention to provide a method for the detection of the genes.

It is still a further object of the present invention to provide a method for diagnosing exposure to electromagnetic radiation.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a heat map showing relative expression levels of 788 genes which change significantly in expression level upon electromagnetic radiation exposure; and

FIG. 2 is a heat map showing relative expression levels of 40 genes which change significantly in expression level upon electromagnetic radiation exposure.

### DESCRIPTION OF THE PREFERRED EMBODIMENTS

In accordance with an aspect thereof, the present invention pertains to a composition for the detection of a diagnostic marker indicative of exposure to electromagnetic radiation, comprising an agent capable of measuring at an mRNA or protein level the expression level of genes given in Table 6.

The term “Electromagnetic radiation”, as used herein, is a form of energy exhibiting wave like behavior as it travels through space. Electromagnetic radiation has both electric and magnetic field components. Electromagnetic radiation is formed when an electric field couples with a magnetic field. Electromagnetic radiation has three elements including wavelength, amplitude and wave form, and carries electrophoton energy that may be imparted to matter with which it interacts. With the shorter wavelengths, the electromagnetic radiation carries larger energy. The wavelength is the distance over which the wave's shape repeats. The electromagnetic radiation is classified according to the frequency of its wave. In order of increasing frequency and decreasing wavelength, these are extremely low frequency, long waves, longitudinal waves, short waves, very high frequency, microwaves, infrared radiation, visible light (laser included), ultraviolet radiation, X-rays and gamma rays. Frequency is the number of occurrences of a repeating event per unit time. The unit of frequency is the hertz. Frequency is in inverse proportion to wavelength. As used herein, the term “electromagnetic radiation” or “electromagnetic wave” is intended to refer to radiofrequency radiation in the range of 100 kHz to 300 GHz, which is widely used in daily life, such as in TV, hand-held phones, radio broadcasting, communication, etc.

The diagnostic marker of electromagnetic radiation exposure in accordance with the present invention may be useful for monitoring and determining the exposure to electromagnetic waves in daily life. In an embodiment of the present invention, 1762.5 MHz RF radiation was employed at a 60 W/kg SAR (specific absorption rate) in the diagnosis of exposure to electromagnetic waves. Thus, exposure to RF radiation at 60 W/kg or higher SAR (specific absorption rate) can be diagnosed in accordance with the present invention.

The term “diagnosis”, as used herein, means the identification of pathological histories or features, and is intended, for the purpose of the present invention, to refer to identify whether a subject was exposed to electromagnetic radiation.

The term “diagnostic marker”, “marker for diagnosis”, or “diagnosis marker”, as used herein, is intended to refer to a material which is capable of discriminating between electromagnetic radiation-exposed cells and normal cells and which increases or decreases in expression level in electromagnetic radiation-exposed cells compared to normal cells. Organic biomolecules such as polypeptides, nucleic acids (e.g., mRNA, etc.), lipids, glycolipids, glycoproteins, etc., fall within the scope of the diagnostic marker. For the purpose of the present invention, the markers which characteristically change in expression level in electromagnetic radiation-exposed cells, compared to normal cells, include genes of GenBank Nos.: NM_{—}006933, NM_{—}002214, NM_{—}020422, NM_{—}018018, NM_{—}001039966, NM_{—}001135599, NM_{—}000867, NM_{—}012098, NM_{—}000287, NM_{—}000593, NM_{—}018370, NM_{—}006702, NM_{—}006645, NM_{—}030941, NM_{—}004540, NM_{—}001034194, NM_{—}001280, BC063625, NM_{—}017858, NM_{—}001099286, NM_{—}005915, NM_{—}005320, NM_{—}130398, NM_{—}004153, NR_{—}002562, NM_{—}022170, NM_{—}004900, NM_{—}003524, NM_{—}182751, NR_{—}002559, NR_{—}002564, NM_{—}006743, NR—002561, NM_{—}003504, NM_{—}003521, NM_{—}002915, NM_{—}005325, NR_{—}002612, NR_{—}002563 and NR_{—}002565 or proteins encoded thereby.

Little is known about the correlation between the functions of the genes and electromagnetic radiation exposure. In the present invention, the genes are proven to be useful as diagnostic markers with regard to RF radiation exposure, as will be illustrated below. For this, total mRNA was isolated from human normal fibroblast WI-38 cells exposed previously to RF radiation, and used to synthesize cDNA which was then labeled with biotin. The labeled cDNA was hybridized with 3GeneChip Human Gene 1.0 ST Array chip and fluorostained with streptavidin-phycoerythrin or biotinylated anti-streptavidin antibody. Differences in gene expression pattern were analyzed by scanning data of the fluorescent images.

As a result of the analysis, 788 genes showed significant changes in expression level: an increase of expression level was detected in 358 genes while the remaining 430 decreased in expression level. The genes of increased expression levels were found to be involved mainly in “negative regulation of developmental process/organ morphogenesis,” “response to protein stimulus,” and “developmental process” (Table 1), being in connection with “Antigen processing and presentation,” “MAPK signaling pathway,” and “Notch signaling pathway” (Table 2). On the other hand, the genes of decreased expression levels were implicated mainly in “cell cycle,” “chromosome organization and biogenesis,” and “response to DNA damage stimulus” (Table 3), as well as being responsible for “cell cycle pathway,” and “DNA polymerase/pyrimidine-, purine-metabolic pathway” (Table 4).

A moderated t-test was conducted with the data of the differentially expressed genes to arrange the genes in the increasing order of p value. Each sample of the genes selected for low p values was predicted using the “leave-one-out” method. Only for samples of 35-40 genes, the prediction error rate was found to be 0% in all used algorithms (Table 5).

In addition, 40 genes were divided into eight samples which were further sub-divided into one test set and seven training set after which a t-test was conducted with the groups. Pre-validation indicated that Diagonal Linear Discriminant Analysis, and support vector machine are the most effective (Table 7).

As used herein, the term “an agent capable of measuring at the mRNA or protein level the expression level of genes” is intended to refer to a molecule which, when reacted with the mRNAs or proteins of the genes given in Table 6, can furnish information about the expression level of the genes. Preferably, the agent is an antibody to the markers or a primer or probe specific for the markers.

The expression levels of the genes of Table 6, that is, the genes of GenBank Nos.: NM_{—}006933, NM_{—}002214, NM_{—}020422, NM_{—}018018, NM_{—}001039966, NM_{—}001135599, NM_{—}000867, NM_{—}012098, NM_{—}000287, NM_{—}000593, NM_{—}018370, NM_{—}006702, NM_{—}006645, NM_{—}030941, NM_{—}004540, NM_{—}001034194, NM_{—}001280, BC063625, NM_{—}017858, NM_{—}001099286, NM_{—}005915, NM_{—}005320, NM_{—}130398, NM_{—}004153, NR_{—}002562, NM_{—}022170, NM_{—}004900, NM_{—}003524, NM_{—}182751, NR_{—}002559, NR_{—}002564, NM_{—}006743, NR_{—}002561, NM_{—}003504, NM_{—}003521, NM_{—}002915, NM_{—}005325, NR_{—}002612, NR_{—}002563 and NR_{—}002565, can be determined by measuring quantities of their mRNAs or proteins.

The term “measurement of the mRNA expression level” is intended to refer to the process of determining the presence and expression level of the mRNA of a marker gene of interest in a biological sample, thereby diagnosing exposure to electromagnetic radiation. It is determined by measuring the quantity of mRNA in the sample. Examples of the assay methods useful for the measurement of mRNA expression level include RT-PCR, competitive RT-PCR, real-time RT-PCR, RNase protection assay (RPA), northern blotting, and DNA microarray chip, but are not limited thereto. The agent capable of quantitatively measuring a gene at an mRNA level is preferably a pair of primers or a probe. Because the sequences of the genes can be registered in the GenBank, primers or probes for amplifying certain ranges of the genes can be designed based on the sequences.

The term “primer,” as used herein, refers to a short strand of nucleic acid sequence which can form base pairings with a complementary template and has a free 3′-hydroxyl group serving as a starting point for template replication. DNA synthesis can start with a template and suitable primers in the presence of a polymerase (e.g., DNA polymerase or reverse trascriptase) under the proper conditions of buffer, reagents, temperatures, four kinds of NTPs, etc. In an embodiment of the present invention, a marker gene is amplified by PCR using a set of sense and antisense primers so as to diagnose electromagnetic radiation exposure. PCR conditions and lengths of sense and antisense primers may be modulated by those skilled in the art.

The term “probe”, as used herein, is intended to refer to a nucleic acid fragment, such as a DNA or RNA fragment, ones to hundreds of bases long, which can form base pairings specifically with mRNA. It may be labeled to detect the presence or absence of a target mRNA. The probe may be constructed in the form of an oligonucleotide probe, a single-stranded DNA probe, a double-stranded DNA probe, or an RNA probe. In an embodiment of the present invention, hybridization between a marker polynucleotide and a complementary probe allows the diagnosis of RF radiation exposure. Choice of suitable probes and conditions for hybridizations may be modulated by those skilled in the art.

The primers or probes of the present invention can be chemically synthesized using a phosphoramidite solid-phase method or another well-known method. Also, the primers or probes may be modified using well-known methods. Non-limiting examples of the modification include methylation, capping, substitution with at least one analogue, and intemucleosidic modification, for example, modification of non-charged linkers (e.g., methylphosphonate, phosphotriester, phosphoroamidate, carbamate, etc.) or charged linkers (e.g., phosphorothioate, phosphorodithioate, etc.) at intemucleosidic sites.

As used herein, the term “measurement of protein expression level” is intended to refer to the process of determining the presence and expression level of a protein encoded by a marker gene of interest in a biological sample, thereby diagnosing exposure to electromagnetic radiation. It is determined by measuring the quantity of the protein encoded by the gene, typically using an antibody to the protein. Examples of the assay methods useful for the measurement of protein expression level include Western blotting, ELISA (enzyme linked immunosorbent assay), radioimmunoassay, radioimmunodiffusion, Ouchterlony immunodiffusion, rocket immunoelectrophoresis, histoimmunostaining, immunoprecipitation assay, complement fixation assay, FACS, and protein chip, but are not limited thereto.

The agent capable of measuring a gene at the protein level is preferably an antibody. The term “antibody,” as used herein, refers to a specialized protein molecule which is specifically directed toward an epitope. In the context of the purpose of the present invention, this term is limited to the antibody which binds specifically to a marker protein of the present invention. The antibody can be prepared using a protein encoded by a marker gene. In a typical method, the market gene is cloned in an expression vector, and the protein is expressed from the vector. Partial peptides derived from the protein may be available. They may be at least 7 amino acids long, preferably 9 amino acids long, and more preferably 12 amino acids long.

No particular limitations are imparted to the form of the antibody. Provided that it has the ability to bind to an antigen, any antibody may be used in the present invention. Polyclonal antibodies, monoclonal antibodies, and fragments thereof and immunoglobulin antibodies fall within the range of the antibody of the present invention. Also, special antibodies, such as humanized antibodies, are among the antibodies of the present invention.

The antibodies useful in detecting diagnostic markers for the diagnosis of exposure to electromagnetic radiation comprise functional fragments of antibody molecules as well as intact antibodies composed of two full-length light chains and two full-length heavy chains. The functional fragments of antibody molecules mean fragments retaining at least antigen-binding functionality, and include Fab, F(ab′), F(ab′)₂ and Fv.

In accordance with a further aspect thereof, the present invention pertains to a kit for diagnosing exposure to electromagnetic radiation, comprising the composition for the detection of a diagnostic marker indicative of exposure to electromagnetic radiation.

The kit can diagnose exposure to electromagnetic radiation by measuring the expression levels of mRNA or protein of marker genes. The kit of the present invention may comprise primers or probes for measuring the expression level of diagnostic markers, antibodies selectively recognizing the markers, one or more components, solutions and/or factors suitable for analysis.

For instance, the kit may be designed to measure the expression of the marker genes at the mRNA level by RT-PCR. Such an RT-PCR kit may comprise elements necessary for RT-PCR, including a pair of primers specific for each of the marker genes, test tubes or other suitable containers, reaction buffers, dNTPs, enzymes such as Tag-polymerase and reverse transcriptase, a DNase inhibitor, an RNase inhibitor, DEPC-water, sterile water, and so forth.

Alternatively, the kit may be designed to measure the expression of the marker genes at the protein level. In this context, it may comprise antibodies and elements necessary for the immunological detection of the antibodies, including a matrix, buffer, coloring enzyme- or fluorescent-labeled secondary antibody, and a coloring substrate. Examples of the matrix include a nitrocellulose membrane, a 96-well plate made of polyvinyl resin or polystyrene resin, and slide glass. Among the coloring enzymes are peroxidase and alkaline phosphatase. FITC or RITC may be used as a fluorescent. ABTS (2,2′-azino-bis-(3-ethylbenzothiazoline-6-sulfonic acid)) or OPD(o-phenylenediamine), or TMB (tetramethyl benzidine) is suitable as the coloring substrate.

In addition, the kit of the present invention may comprise elements necessary for DNA microarray chip analysis. For example, a DNA microarray chip kit may comprise a substrate on which cDNAs of marker genes or their oligonucleotide segments are arranged along with a quantitative control gene or its cDNA. In detail, the DNA microarray chip may comprise the genes of Table 6, or their oligonucleotide segments or their complementary strand molecules which are clustered on a substrate. Each of the oligonucleotide segments or complementary strand molecules may be comprised of 18 to 30 nucleotides and preferably 20 to 25 nucleotides of the marker genes. The DNA microarray chip may be constructed by a well-known method using the marker genes of the present invention. For example, the marker genes may be immobilized onto the substrate of the DNA chip using a piezoelectric micropipetting technique or a pin-type spotter. The substrate of the DNA microarray chip is coated preferably with a functional group selected from a group consisting of amino-silane, poly-L-lysine and aldehyde, but the present invention is not limited by the examples. The substrate may be preferably selected from a group consisting of slide glass, plastic, metal, silicon, a nylon membrane, and a nitrocellulose membrane, but the present invention is not limited to these.

In accordance with a further aspect thereof, the present invention pertains to a method for detecting a diagnostic maker gene indicative of exposure to electromagnetic radiation, comprising measuring at an mRNA or protein level the expression level of the marker genes in a sample from a subject; and comparing the expression level of the genes with that of corresponding genes from a normal control, and to a method for diagnosing exposure to electromagnetic radiation, using the detection method.

In detail, the expression levels of the mRNA or proteins corresponding to the marker genes can be measured. The mRNAs or proteins can be isolated from a biological sample using a well-known method.

The term “sample from a subject,” as used herein, is intended to include tissues, cells, whole blood, sera, plasma, sputum, saliva, cerebrospinal fluid and urine in which the maker genes show differential expression levels. In an embodiment of the present invention, fibroblast WI-38 was used as a sample.

Comparison of the expression levels of the marker genes between a normal control and a subject of interest, that is, a subject suspected of RF radiation exposure, makes it possible to determine whether the subject suspected of RF radiation exposure was practically exposed to RF radiation. For example, expression levels of the marker genes of respective samples from a subject suspected of RF radiation exposure and a normal control are measured and then the expression levels are compared to each other. When genes with GenBank Nos.: NM_{—}006933, NM_{—}002214, NM_{—}020422, NM_{—}018018, NM_{—}001039966, NM_{—}001135599, NM_{—}000867, NM_{—}012098, NM_{—}000287, NM_{—}000593, NM_{—}018370, NM_{—}006702 and NM_{—}006645 of the marker genes of the present invention are higher in expression level in a subject suspected of electromagnetic radiation exposure than in a normal control, the subject may be predicted to be exposed to electromagnetic radiation.

On the other hand, when genes with GenBank Nos: NM_{—}030941, NM_{—}004540, NM_{—}001034194, NM_{—}001280, BC063625, NM_{—}017858, NM_{—}001099286, NM_{—}005915, NM_{—}005320, NM_{—}130398, NM_{—}004153, NR_{—}002562, NM_{—}022170, NM_{—}004900, NM_{—}003524, NM_{—}182751, NR_{—}002559, NR_{—}002564, NM_{—}006743, NR_{—}002561, NM_{—}003504, NM_{—}003521, NM_{—}002915, NM_{—}005325, NR_{—}002612, NR_{—}002563 and NR_{—}002565 of the marker genes of the present invention are lower in expression level in a subject suspected of electromagnetic radiation exposure than in a normal control, the subject may be predicted to be exposed to electromagnetic radiation.

Assay methods of measuring mRNA levels may be exemplified by RT-PCR, competitive RT-PCR, real-time RT-PCR, reverse transcriptase polymerization, RNase protection assay, Northern blotting, and DNA microarray chip, but no specific method must be used in the present invention and as such does not limit the confines of the present invention. By the methods, the expression levels of mRNA of the marker gene can be compared between a normal group and a suspected group. Also, significant changes in the mRNA level of marker genes allow the diagnosis of the practical exposure of suspected subjects to electromagnetic radiation.

The measurement of mRNA expression level can be achieved preferably using RT-PCR with primers specific for marker genes or using a DNA microarray chip.

After RT-PCR, the mRNA expression levels of marker genes diagnostic of exposure to electromagnetic waves are analyzed by examining the patterns and thicknesses of the bands separated upon electrophoresis. The mRNA expression levels are compared with those of a control so as to simply diagnose electromagnetic radiation exposure.

As for the DNA microarray chip, it comprises the marker genes or their fragments that are very densely arranged on a substrate such as a glass plate. The mRNA isolated from a sample is used to synthesize cDNA probes labeled at an end or at an internal site with a fluorescent material. The cDNA probes are hybridized with the DNA chip so that electromagnetic radiation exposure can be diagnosed. In detail, this can be conducted by: isolating mRNAs of the marker genes of the present invention from samples from both a subject and a normal control; synthesizing cDNAs from the mRNAs, with respective fluorescent material incorporated thereinto; hybridizing the fluorescent-labeled cDNAs with a DNA microarray chip; and analyzing the hybridized DNA microarray chip to compare mRNA expression levels of the marker genes of the present invention between the subject and the normal control.

Examples of the fluorescent materials useful in the present invention include, but are not limited to, Cy3, Cy5, FITC (poly L-lysine-fluorescein isothiocyanate), RITC (rhodamine-B-isothiocyanate) and rhodamine. Any well-known fluorescent material may be used in the present invention. 36 k Human V4.0 OpArray oligomicroarray (Operon, Germany) or whole human genome oligo microarray (Agilent, USA) is suitable as the microarray chip, but does not limit the present invention in any way. So long as it is loaded with the commonly up-regulated or down-regulated genes, any DNA chip may be employed.

Assay methods of measuring protein levels may be exemplified by Western blotting, ELISA, radioimmunoassay, radioimmunodiffusion, Ouchterlony immunodiffusion, rocket immunoelectrophoresis, histoimmunostaining, immunoprecipitation assay, complement fixation assay, FACS, and protein chip, but are not limited thereto. By this assay method, for example, the quantities of the formed antigen-antibody complexes of a RF radiation exposure-suspected subject are compared with a normal control. Significant increases or decreases in the protein expression levels of the marker genes provide important information about the diagnosis of practical exposure to electromagnetic radiation.

As used herein, the term “antigen-antibody complex” means a conjugate of a maker protein and an antibody specific therefor. The formation of an antigen-antibody complex may be quantitatively determined by measuring the signal intensity of the detection label.

The measurement of protein expression level may also be achieved using ELISA. Examples to of ELISA include direct ELISA in which a labeled antibody immobilized onto a solid support is used to recognize an antigen, indirect ELISA in which a labeled antibody is used to recognize a captured antibody immobilized on a solid support which is complexed with an antigen, direct sandwich ELISA in which an antibody is used to recognize an antigen captured by another antibody immobilized onto a solid support, and indirect sandwich ELISA in which a secondary antibody is used to recognize an antibody which captures an antigen complexed with a different antibody immobilized onto a solid support. For example, an antibody is immobilized onto a solid support and is reacted with a sample to form an antigen-antibody. Then, a labeled antibody specific for the antigen is allowed to capture the antigen of the complex, followed by enzymatic color development. Alternatively, an antibody specific for the antigen is allowed to capture the antigen of the complex and then is recognized by a labeled secondary antibody, followed by enzymatic color development. The formation of the complex of a marker protein with an antibody can thus be quantitatively measured so as to diagnose electromagnetic radiation exposure.

In another embodiment, the measurement of protein expression level is achieved using Western blotting. Proteins are isolated from a sample, separated according to size by electrophoresis, transferred onto a nitrocellulose membrane, and reacted with an antibody to form an antigen-antibody complex. The quantity of the complex is measured using a labeled secondary antibody. The expression level of the protein encoded by a marker gene provides important information about the diagnosis of electromagnetic radiation exposure. The detection method is conducted by measuring the expression levels of the marker proteins in the control and the electromagnetic radiation exposure-suspected subject. The expression levels of mRNA or protein may be represented by the different marker protein expression levels between these two on an absolute (e.g., μg/ml) or relative (e.g., relative intensity of signal) scale.

In another embodiment, the protein expression level is determined by a histoimmunostaining method using at least one antibody to the marker. A tissue taken from an electromagnetic radiation exposure-suspected subject is fixed and embedded in paraffin. The paraffin block is cut into slices several μm thick which are then placed on glass slides. An antibody is applied to the tissue slices, followed by washing off the unreacted antibodies. Thereafter, the antibody is conjugated with a detection label which is then observed under a microscope.

A protein chip in which one or more antibodies to the marker are arranged at predetermined positions and fixed at a high density on a substrate may be used to measure the protein expression level. In this regard, proteins isolated from a sample are hybridized with the protein chip to form antigen-antibody complexes. The formation of the antigen-antibody complex can be thus quantitatively read so as to diagnose electromagnetic radiation exposure.

As described above, the diagnostic markers in accordance with the present invention are very useful for monitoring and diagnosing exposure to electromagnetic fields, and can be used as instruments by which physiological mechanisms incurred upon electromagnetic radiation exposure are examined.

A better understanding of the present invention may be obtained through the following examples which are set forth to illustrate, but are not to be construed as the limit of the present invention.

### EXAMPLE 1.

### Experiment Methods

1-1. Electromagnetic Radiation Exposure

Human normal fibroblast WI-38 cells were exposed for 24 hrs to 1762.5 MHz radiation at a 60 W/kg specific absorption ratio (SAR). Normal WI-38 cells which were incubated for 24 hrs in a 37° C. incubator without RF radiation exposure were used as a control.

1-2. mRNA Isolation

Total RNA was isolated using an RNeasy Mini kit (Qiagen GmbH, Hilden, Germany). The purity and integrity of the isolated RNA were determined using a Nanodrop spectrometer (NanoDrop Technologies, Wilmington, Del., USA) and an Agilent bioanalyzer (Agilent Technologies, Santa Clara, Calif., USA), respectively.

1-3. mRNA Microarray

The chip used was GeneChip Human Gene 1.0 ST Array of Affymetrix. Of the total RNA, 100 ng was amplified using RT-PCR and the amplification product of the RNA was processed and labeled with biotin according to the Affymetrix Genechip Whole Transcript(Wi) Sense Target Labeling assay. Then, 5.5 μg of the biotin-labeled sense DNA was hybridized to Affymetrix Human Gene 1.0 ST arrays and immunostained against streptavidin-phycoerythrin or biotinylated anti-streptavidin antibody according to a protocol, followed by scanning.

1-4. mRNA Microarray Analysis and Prediction of Electromagnetic Radiation Exposure

a. Selection of Algorithms and Genes to be Used in Prediction Algorithm

Samples were divided to RF radiation-exposed and RF radiation-non-exposed groups. A moderated t-test was conducted to examine whether there was a difference in mRNA expression level between the two groups. Genes were arranged in the increasing order of p value. A classification algorithm was applied to gene groups starting from the top five genes, with an increase in the number of genes of subsequent p value order by five. Various supervised machine learning algorithms were conducted to select the algorithm showing the highest prediction accuracy. Used algorithms were as follows: k-Nearest Neighbor, Linear Discriminant Analysis (LDA), Diagonal Linear Discriminant Analysis, Random Forest, naive Bayes, Neural Networks, Support Vector Machines (SVM), Generalized Linear Models (GLM)

b. Pre-Validation

An evaluation was made by Leave-One-Out(LOO) validation. Samples were divided into eight groups: one was used as a test set while the other seven were used as training sets. Only the training sets were used to select genes which would be used for the prediction of RF radiation exposure by moderated t-test. They were sub-divided into RF radiation-exposed and non-exposed groups, followed by the application of moderated t-test and the genes were arranged in increasing order of p value. As many genes as the orders thereof were selected. The selected genes were applied to a supervised machine learning algorithm to predict the exposure of the test set to RF radiation. This procedure was repeated eight times to obtain prediction results as concerns the exposure of each sample to RF radiation. Taken together, these results were used to calculate error rates.

### EXAMPLE 2

### Test Results

2-1. Genes Changed in Expression Level upon RF Radiation Exposure

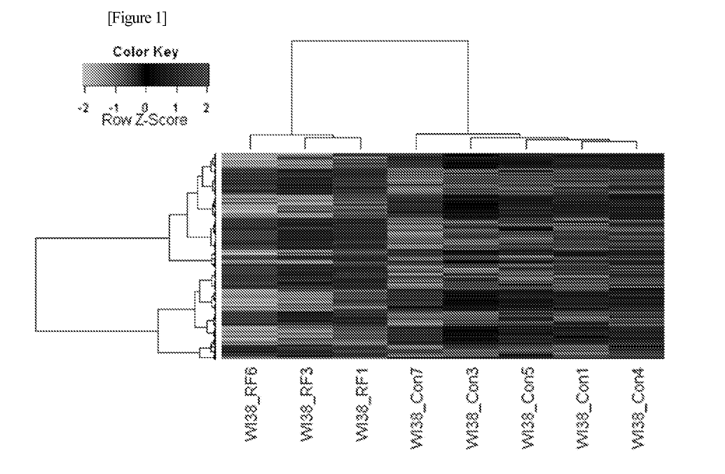

Upon RF radiation exposure, 788 genes showed significant changes in expression level: an increase of expression level was detected in 358 genes while the remaining 430 decreased in expression level. Multiple testing corrections were performed using the Benjamini-Hochberg False Discovery Rate (BH FDR) method with increasing type I error rates, with significance after controlling for an BH FDR of 5%. The relative expression levels of the 788 genes are depicted in the heat map of FIG. 1.

As a result of analysis, the genes of increased expression levels were found to be involved mainly in “negative regulation of developmental process/organ morphogenesis,” “response to protein stimulus,” and “developmental process” (Table 1), being in connection with “Antigen processing and presentation,” “MAPK signaling pathway,” and “Notch signaling pathway” (Table 2).

TABLE 1

| Functional | | | | | BH FDR |
|---|---|---|---|---|---|
| group | Term | Count | % | P value | P value |
| Functional | negative regulation of developmental process | 9 | 2.89% | 9.39E−05 | 0.15 |
| Group 1 | negative regulation of cell differentiation | 8 | 2.57% | 1.75E−04 | 0.17 |
| | regulation of developmental process | 14 | 4.50% | 2.78E−04 | 0.19 |
| | regulation of cell differentiation | 10 | 3.22% | 0.001 | 0.30 |
| Functional | organ morphogenesis | 19 | 6.11% | 1.23E−04 | 0.15 |
| Group 2 | angiogenesis | 10 | 3.22% | 4.82E−04 | 0.27 |
| | anatomical structure formation | 11 | 3.54% | 6.06E−04 | 0.25 |
| | blood vessel morphogenesis | 10 | 3.22% | 0.0014 | 0.30 |
| | blood vessel development | 10 | 3.22% | 0.0033 | 0.46 |
| | vasculature development | 10 | 3.22% | 0.0036 | 0.48 |
| | muscle cell differentiation | 5 | 1.61% | 0.0088 | 0.69 |
| | regulation of angiogenesis | 4 | 1.29% | 0.026 | 0.88 |
| Functional | protein folding | 14 | 4.50% | 5.04E−04 | 0.25 |
| Group 3 | response to protein stimulus | 8 | 2.57% | 7.60E−04 | 0.26 |
| | response to unfolded protein | 8 | 2.57% | 7.60E−04 | 0.26 |
| | response to biotic stimulus | 12 | 3.86% | 0.012 | 0.78 |
| | response to chemical stimulus | 18 | 5.79% | 0.017 | 0.84 |
| Functional | system development | 48 | 15.43% | 2.57E−04 | 0.20 |
| Group 4 | anatomical structure morphogenesis | 34 | 10.93% | 5.58E−04 | 0.25 |
| | multicellular organismal development | 57 | 18.33% | 0.0011 | 0.29 |
| | anatomical structure development | 53 | 17.04% | 0.0013 | 0.29 |
| | organ development | 35 | 11.25% | 0.0023 | 0.39 |
| | developmental process | 70 | 22.51% | 0.0058 | 0.58 |
| | cellular developmental process | 44 | 14.15% | 0.0061 | 0.59 |
| | cell differentiation | 44 | 14.15% | 0.0061 | 0.59 |
| Functional | regulation of cell proliferation | 19 | 6.11% | 0.0011 | 0.29 |
| Group 5 | negative regulation of cellular process | 31 | 9.97% | 0.0042 | 0.51 |
| | negative regulation of cell proliferation | 10 | 3.22% | 0.016 | 0.83 |
| | cell proliferation | 21 | 6.75% | 0.03 | 0.90 |
| Functional | signal transduction | 79 | 25.40% | 0.0051 | 0.57 |
| Group 6 | cell communication | 85 | 27.33% | 0.0058 | 0.59 |
| | intracellular signaling cascade | 36 | 11.58% | 0.014 | 0.80 |
| Functional | lipid metabolic process | 24 | 7.72% | 0.0026 | 0.43 |
| Group 7 | membrane lipid metabolic process | 9 | 2.89% | 0.022 | 0.88 |
| | cellular lipid metabolic process | 18 | 5.79% | 0.023 | 0.88 |
| Functional | system development | 48 | 15.43% | 2.57E−04 | 0.20 |
| Group 8 | negative regulation of biological process | 33 | 10.61% | 0.0022 | 0.40 |
| | cell development | 34 | 10.93% | 0.0027 | 0.42 |
| | negative regulation of cellular process | 31 | 9.97% | 0.0042 | 0.51 |
| | developmental process | 70 | 22.51% | 0.0058 | 0.58 |
| | cellular developmental process | 44 | 14.15% | 0.0061 | 0.59 |
| | cell differentiation | 44 | 14.15% | 0.0061 | 0.59 |
| | negative regulation of apoptosis | 10 | 3.22% | 0.012 | 0.77 |
| | regulation of apoptosis | 16 | 5.14% | 0.026 | 0.88 |
| | cell death | 21 | 6.75% | 0.045 | 0.95 |
| | apoptosis | 20 | 6.43% | 0.046 | 0.95 |
| Functional | positive regulation of cellular process | 30 | 9.65% | 0.0012 | 0.30 |
| Group 9 | positive regulation of biological process | 31 | 9.97% | 0.003 | 0.44 |
| | positive regulation of metabolic process | 16 | 5.14% | 0.0053 | 0.57 |
| | positive regulation of cellular metabolic process | 14 | 4.50% | 0.016 | 0.84 |
| | positive regulation of nucleobase, nucleoside, nucleotide and nucleic | 12 | 3.86% | 0.019 | 0.85 |
| | acid metabolic process | | | | |
| | positive regulation of transcription | 11 | 3.54% | 0.037 | 0.93 |
| | regulation of transcription from RNA polymerase II promoter | 14 | 4.50% | 0.042 | 0.94 |
| Functional | biological regulation | 119 | 38.26% | 3.27E−06 | 0.02 |
| Group 10 | regulation of biological process | 106 | 34.08% | 5.19E−05 | 0.13 |
| | regulation of cellular process | 95 | 30.55% | 7.15E−04 | 0.27 |
| | transcription from RNA polymerase II promoter | 18 | 5.79% | 0.04 | 0.94 |

TABLE 2

| KEGG | | | BH | | |
|---|---|---|---|---|---|
| PATHWAY | % | PValue | FDR | Gene | Gene Full Name |
| Antigen processing and | 1.93% | 0.021 | 0.99 | RFX5 | REGULATORY FACTOR X, 5 (INFLUENCES HLA CLASS II EXPRESSION) |
| presentation | | | | TAP1 | TRANSPORTER 1, ATP-BINDING CASSETTE, SUB-FAMILY B (MDR/TAP) |
| | | | | HSPA1A | HEAT SHOCK 70 KDA PROTEIN 1A |
| | | | | HSPA1B | HEAT SHOCK 70 KDA PROTEIN 1A |
| | | | | HSPA1L | HEAT SHOCK 70 KDA PROTEIN 1-LIKE |
| | | | | HSPA2 | HEAT SHOCK 70 KDA PROTEIN 2 |
| | | | | TAPBP | TAP BINDING PROTEIN (TAPASIN) |
| MAPK signaling | 3.22% | 0.071 | 1.00 | CACNB3 | CALCIUM CHANNEL, VOLTAGE-DEPENDENT, BETA 3 SUBUNIT |
| pathway | | | | IL1R1 | INTERLEUKIN 1 RECEPTOR, TYPE I |
| | | | | MAP2K6 | MITOGEN-ACTIVATED PROTEIN KINASE KINASE 6 |
| | | | | ARRB1 | ARRESTIN, BETA 1 |
| | | | | FOS | V-FOS FBJ MURINE OSTEOSARCOMA VIRAL ONCOGENE HOMOLOG |
| | | | | SOS1 | SON OF SEVENLESS HOMOLOG 1 (DROSOPHILA) |
| | | | | MAP3K12 | MITOGEN-ACTIVATED PROTEIN KINASE KINASE KINASE 12 |
| | | | | ECSIT | SIGNALING INTERMEDIATE IN TOLL PATHWAY, EVOLUTIONARILY |
| | | | | | CONSERVED |
| | | | | CDC25B | CELL DIVISION CYCLE 25B |
| | | | | MKNK2 | MAP KINASE INTERACTING SERINE/THREONINE KINASE 2 |
| Notch signaling | 1.29% | 0.067 | 1.00 | JAG1 | JAGGED 1 (ALAGILLE SYNDROME) |
| pathway | | | | CREBBP | CREB BINDING PROTEIN (RUBINSTEIN-TAYBI SYNDROME) |
| | | | | KAT2A | GCN5 GENERAL CONTROL OF AMINO-ACID SYNTHESIS 5-LIKE 2 (YEAST) |
| | | | | DTX3 | DELTEX 3 HOMOLOG (DROSOPHILA) |

On the other hand, the genes of decreased expression levels were implicated mainly in “cell cycle,” “chromosome organization and biogenesis,” and “response to DNA damage stimulus” (Table 3), as well as being responsible for “cell cycle pathway,” and “DNA polymerase/pyrimidine-, purine-metabolic pathway” (Table 4).

TABLE 3

| Annotation | | | | | BH FDR |
|---|---|---|---|---|---|
| cluster | Term | Count | % | P value | P value |
| Functional | cell cycle | 105 | 28.77% | 3.61E−58 | 1.89E−54 |
| Group 1 | cell cycle phase | 68 | 18.63% | 5.46E−50 | 9.57E−47 |
| | cell cycle process | 90 | 24.66% | 9.88E−50 | 1.30E−46 |
| | M phase | 62 | 16.99% | 9.82E−49 | 1.03E−45 |
| | mitotic cell cycle | 62 | 16.99% | 6.78E−46 | 5.94E−43 |
| | mitosis | 55 | 15.07% | 7.54E−46 | 5.66E−43 |
| | M phase of mitotic cell cycle | 55 | 15.07% | 1.25E−45 | 8.24E−43 |
| | cell division | 49 | 13.42% | 1.91E−36 | 1.11E−33 |
| Functional | chromosome organization and biogenesis | 52 | 14.25% | 1.31E−29 | 6.26E−27 |
| Group 2 | organelle organization and biogenesis | 79 | 21.64% | 4.51E−25 | 1.82E−22 |
| | nucleosome assembly | 24 | 6.58% | 8.61E−21 | 2.38E−18 |
| | chromatin assembly | 25 | 6.85% | 1.35E−20 | 3.55E−18 |
| | chromatin assembly or disassembly | 26 | 7.12% | 6.27E−18 | 1.32E−15 |
| | establishment and/or maintenance of chromatin architecture | 34 | 9.32% | 1.52E−16 | 2.24E−14 |
| | DNA packaging | 34 | 9.32% | 2.66E−16 | 4.32E−14 |
| | protein-DNA complex assembly | 25 | 6.85% | 3.22E−16 | 6.25E−14 |
| | cellular component organization and biogenesis | 94 | 25.75% | 4.60E−11 | 7.11E−09 |
| | macromolecular complex assembly | 33 | 9.04% | 5.60E−09 | 7.01E−07 |
| | cellular component assembly | 34 | 9.32% | 8.82E−09 | 1.08E−06 |
| Functional | response to DNA damage stimulus | 46 | 12.60% | 1.86E−27 | 8.15E−25 |
| Group 3 | DNA repair | 39 | 10.68% | 1.19E−23 | 4.48E−21 |
| | response to endogenous stimulus | 46 | 12.60% | 1.96E−23 | 6.43E−21 |
| | response to stress | 51 | 13.97% | 3.28E−10 | 4.93E−08 |
| | response to stimulus | 63 | 17.26% | 0.14 | 1.00 |
| Functional | cell cycle checkpoint | 20 | 5.48% | 6.19E−19 | 1.48E−16 |
| Group 4 | regulation of mitosis | 17 | 4.66% | 6.72E−14 | 1.18E−11 |
| | mitotic cell cycle checkpoint | 10 | 2.74% | 4.12E−09 | 5.41E−07 |
| Functional | nucleobase, nucleoside, nucleotide and nucleic acid metabolic process | 144 | 39.45% | 2.01E−20 | 5.04E−18 |
| Group 5 | biopolymer metabolic process | 166 | 45.48% | 2.53E−18 | 5.55E−16 |
| | macromolecule metabolic process | 178 | 48.77% | 1.58E−09 | 2.25E−07 |
| | cellular metabolic process | 195 | 53.42% | 3.18E−09 | 4.39E−07 |
| | cellular process | 262 | 71.78% | 3.97E−09 | 5.35E−07 |
| | primary metabolic process | 195 | 53.42% | 4.20E−09 | 5.38E−07 |
| | metabolic process | 202 | 55.34% | 9.41E−07 | 1.01E−04 |
| Functional | chromosome segregation | 17 | 4.66% | 1.11E−14 | 2.01E−12 |
| Group 6 | mitotic sister chromatid segregation | 13 | 3.56% | 8.17E−14 | 1.38E−11 |
| | sister chromatid segregation | 13 | 3.56% | 1.29E−13 | 2.11E−11 |
| | chromosome condensation | 7 | 1.92% | 1.29E−06 | 1.36E−04 |
| | mitotic chromosome condensation | 6 | 1.64% | 6.36E−06 | 6.30E−04 |
| Functional | mitotic cell cycle checkpoint | 10 | 2.74% | 4.12E−09 | 5.41E−07 |
| Group 7 | mitotic cell cycle spindle assembly checkpoint | 4 | 1.10% | 1.84E−04 | 0.013 |
| | spindle checkpoint | 4 | 1.10% | 2.91E−04 | 0.019 |
| Functional | DNA unwinding during replication | 6 | 1.64% | 4.44E−06 | 4.57E−04 |
| Group 8 | DNA duplex unwinding | 6 | 1.64% | 8.88E−06 | 8.64E−04 |
| | DNA geometric change | 6 | 1.64% | 8.88E−06 | 8.64E−04 |
| Functional | spindle organization and biogenesis | 10 | 2.74% | 2.20E−11 | 3.50E−09 |
| Group 9 | phosphoinositide-mediated signaling | 11 | 3.01% | 1.90E−05 | 0.0017 |
| | second-messenger-mediated signaling | 12 | 3.29% | 0.0049 | 0.24 |
| | intracellular signaling cascade | 24 | 6.58% | 0.80 | 1.00 |
| Functional | DNA integrity checkpoint | 9 | 2.47% | 6.53E−08 | 7.63E−06 |
| Group 10 | DNA damage checkpoint | 6 | 1.64% | 1.43E−04 | 0.011 |
| | DNA damage response, signal transduction | 7 | 1.92% | 1.96E−04 | 0.014 |
| | intra-S DNA damage checkpoint | 3 | 0.82% | 0.003 | 0.16 |

TABLE 4

| KEGG | | | | | |
|---|---|---|---|---|---|
| PATHWAY | % | PValue | BH FDR | Gene | Gene Full Name |
| Cell cycle | 7.61% | 1.92E−23 | 3.85E−21 | BUB1B | BUB1 BUDDING UNINHIBITED BY BENZIMIDAZOLES 1 HOMOLOG BETA |
| | | | | CCNA2 | CYCLIN A2 |
| | | | | CDC6 | CDC6 CELL DIVISION CYCLE 6 HOMOLOG (S. CEREVISIAE) |
| | | | | CDC20 | CDC20 CELL DIVISION CYCLE 20 HOMOLOG (S. CEREVISIAE) |
| | | | | CDKN2C | CYCLIN-DEPENDENT KINASE INHIBITOR 2C (P18, INHIBITS CDK4) |
| | | | | CDC2 | CELL DIVISION CYCLE 2. G1 TO S AND G2 TO M |
| | | | | CDC25A | CELL DIVISION CYCLE 25A |
| | | | | MAD2L1 | MAD2 MITOTIC ARREST DEFICIENT-LIKE 1 (YEAST) |
| | | | | MCM3 | MCM3 MINICHROMOSOME MAINTENANCE DEFICIENT 3 (S. CEREVISIAE) |
| | | | | ORC5L | ORIGIN RECOGNITION COMPLEX, SUBUNIT 5-LIKE (YEAST) |
| | | | | RBL1 | RETINOBLASTOMA-LIKE 1 (P107) |
| | | | | CDC7 | CDC7 CELL DIVISION CYCLE 7 (S. CEREVISIAE) |
| | | | | CDC45L | CDC45 CELL DIVISION CYCLE 45-LIKE (S. CEREVISIAE) |
| | | | | ORC1L | ORIGIN RECOGNITION COMPLEX, SUBUNIT 1-LIKE (YEAST) |
| | | | | BUB1 | BUB1 BUDDING UNINHIBITED BY BENZIMIDAZOLES 1 HOMOLOG (YEAST) |
| | | | | MCM2 | MCM2 MINICHROMOSOME MAINTENANCE DEFICIENT 2, MITOTIN |
| | | | | CCNB2 | CYCLIN B2 |
| | | | | BUB3 | BUB3 BUDDING UNINHIBITED BY BENZIMIDAZOLES 3 HOMOLOG (YEAST) |
| | | | | PLK1 | POLO-LIKE KINASE 1 (DROSOPHILA) |
| | | | | MCM4 | MCM4 MINICHROMOSOME MAINTENANCE DEFICIENT 4 (S. CEREVISIAE) |
| | | | | MCM6 | MCM6 MINICHROMOSOME MAINTENANCE DEFICIENT 6 |
| | | | | MCM7 | MCM7 MINICHROMOSOME MAINTENANCE DEFICIENT 7 (S. CEREVISIAE) |
| | | | | MCM5 | MCM5 MINICHROMOSOME MAINTENANCE DEFICIENT 5. CELL DIVISION |
| | | | | | CYCLE 46 (S. CEREVISIAE) |
| | | | | ORC6L | ORIGIN RECOGNITION COMPLEX, SUBUNIT 6 HOMOLOG-LIKE (YEAST) |
| | | | | ANAPC10 | ANAPHASE PROMOTING COMPLEX SUBUNIT 10 |
| | | | | CCNE2 | CYCLIN E2 |
| | | | | ORC3L | ORIGIN RECOGNITION COMPLEX, SUBUNIT 3-LIKE (YEAST) |
| | | | | PKMYT1 | PROTEIN KINASE, MEMBRANE ASSOCIATED TYROSINE/THREONINE 1 |
| DNA | 1.63% | 7.85E−05 | 0.0052 | PRIM1 | PRIMASE, POLYPEPTIDE 1, 49 KDA |
| polymerase | | | | PRIM2 | PRIMASE, POLYPEPTIDE 2A, 58 KDA |
| | | | | POLA2 | POLYMERASE (DNA DIRECTED), ALPHA 2 (70 KD SUBUNIT) |
| | | | | POLE2 | POLYMERASE (DNA DIRECTED), EPSILON 2 (P59 SUBUNIT) |
| | | | | POLD3 | POLYMERASE (DNA-DIRECTED), DELTA 3, ACCESSORY SUBUNIT |
| | | | | POLA1 | POLYMERASE (DNA DIRECTED), ALPHA |
| Purine | 2.99% | 4.36E−04 | 0.022 | DCK | DEOXYCYTIDINE KINASE |
| metabolism | | | | PRIM1 | PRIMASE, POLYPEPTIDE 1, 49 KDA |
| | | | | PRIM2 | PRIMASE, POLYPEPTIDE 2A, 58 KDA |
| | | | | RRM1 | RIBONUCLEOTIDE REDUCTASE M1 POLYPEPTIDE |
| | | | | RRM2 | RIBONUCLEOTIDE REDUCTASE M2 POLYPEPTIDE |
| | | | | POLA2 | POLYMERASE (DNA DIRECTED), ALPHA 2 (70 KD SUBUNIT) |
| | | | | POLE2 | POLYMERASE (DNA DIRECTED), EPSILON 2 (P59 SUBUNIT) |
| | | | | POLD3 | POLYMERASE (DNA-DIRECTED), DELTA 3, ACCESSORY SUBUNIT |
| | | | | ADK | ADENOSINE KINASE |
| | | | | POLA1 | POLYMERASE (DNA DIRECTED), ALPHA |
| | | | | PNPT1 | POLYRIBONUCLEOTIDE NUCLEOTIDYLTRANSFERASE 1 |
| Pyrimidine | 3.26% | 7.29E−07 | 7.33E−05 | DCK | DEOXYCYTIDINE KINASE |
| metabolism | | | | PRIM1 | PRIMASE, POLYPEPTIDE 1, 49 KDA |
| | | | | PRIM2 | PRIMASE, POLYPEPTIDE 2A, 58 KDA |
| | | | | RRM1 | RIBONUCLEOTIDE REDUCTASE M1 POLYPEPTIDE |
| | | | | RRM2 | RIBONUCLEOTIDE REDUCTASE M2 POLYPEPTIDE |
| | | | | DHODH | DIHYDROOROTATE DEHYDROGENASE |
| | | | | CTPS | CTP SYNTHASE |
| | | | | POLA2 | POLYMERASE (DNA DIRECTED), ALPHA 2 (70 KD SUBUNIT) |
| | | | | POLE2 | POLYMERASE (DNA DIRECTED), EPSILON 2 (P59 SUBUNIT) |
| | | | | POLD3 | POLYMERASE (DNA-DIRECTED), DELTA 3, ACCESSORY SUBUNIT |
| | | | | POLA1 | POLYMERASE (DNA DIRECTED), ALPHA |
| | | | | PNPT1 | POLYRIBONUCLEOTIDE NUCLEOTIDYLTRANSFERASE 1 |
| Glycosylphospha- | 0.82% | 0.062 | 0.92 | PIGL | PHOSPHATIDYLINOSITOL GLYCAN, CLASS L |
| tidylinositol | | | | PIGA | PHOSPHATIDYLINOSITOL GLYCAN, CLASS A (PAROXYSMAL |
| (GPI)-anchor | | | | | NOCTURNAL HEMOGLOBINURIA) |
| biosynthesis | | | | PIGW | PHOSPHATIDYLINOSITOL GLYCAN, CLASS W |

2-2. Prediction Using Genes Selected with Total Data

A moderated t-test was conducted with the total data [RF radiation-exposed group (n=3) and non-exposed group (n=5)] to arrange the genes in the increasing order of p value and each sample was predicted using the “leave-one-out” method. Only for samples of 35-40 genes, as is apparent from the data of Table 5, the prediction error rate was found to be 0% in all used algorithms. As seen in Table 5, when 40 genes were applied to eight supervised machine learning algorithms [k-Nearest Neighbor,

Linear Discriminant Analysis (LDA), Diagonal Linear Discriminant Analysis, Random Forest, naive Bayes, Neural Networks, Support Vector Machines (SVM), Generalized Linear Models (GLM)], 100% prediction accuracy was obtained.

TABLE 5

| Algorithm of supervised | Prediction | Number of selected features (genes) by moderated t test | | | | | | | | | | |
|---|---|---|---|---|---|---|---|---|---|---|---|---|
| machine learning | error rate | 5 | 10 | 15 | 20 | 25 | 30 | 35 | 40 | 45 | 50 | 75 |
| k-Nearest Neighbour | Total | 0.00 | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Linear Discriminant | Total | 0.00 | 0.00 | 0.00 | 0.00 | 0.00 | 0.00 | 0.00 | 0.00 | 0.00 | 0.00 | 0.00 |
| Analysis (LDA) | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Diagonal Linear | Total | 0.00 | 0.00 | 0.00 | 0.00 | 0.00 | 0.00 | 0.00 | 0.00 | 0.00 | 0.00 | 0.00 |
| Discriminant Analysis | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Random Forest | Total | 0.00 | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| naive Bayes | Total | 0.00 | 0.00 | 0.13 | 0.13 | 0.13 | 0.13 | 0.00 | 0.00 | 0.00 | 0.00 | 0.00 |
| | RF_Heat | 0.00 | 0.00 | 0.33 | 0.33 | 0.33 | 0.33 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Neural Networks | Total | 0.00 | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Support Vector Machines | Total | 0.00 | 0.00 | 0.00 | 0.00 | 0.00 | 0.00 | 0.00 | 0.00 | 0.00 | 0.00 | 0.00 |
| (SVM) | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Generalized Linear Models | Total | 0.00 | 0.13 | 0.25 | 0.25 | 0.00 | 0.00 | 0.00 | 0.00 | 0.13 | 0.13 | 0.13 |
| (GLM) | RF_Heat | 0.00 | 0.33 | 0.67 | 0.33 | 0.00 | 0.00 | 0.00 | 0.00 | 0.33 | 0.33 | 0.00 |
| | Control | 0.00 | 0.00 | 0.00 | 0.20 | 0.00 | 0.00 | 0.00 | 0.00 | 0.00 | 0.00 | 0.20 |

| Algorithm of supervised | Prediction | Number of selected features (genes) by moderated t test | | | | | | | | | |
|---|---|---|---|---|---|---|---|---|---|---|---|
| machine learning | error rate | 100 | 125 | 150 | 175 | 200 | 225 | 250 | 275 | 300 | 325 |
| k-Nearest Neighbour | Total | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| Linear Discriminant | Total | 0.00 | 0.00 | 0.00 | 0.00 | 0.00 | 0.00 | 0.00 | 0.00 | 0.00 | 0.00 |
| Analysis (LDA) | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| Diagonal Linear | Total | 0.00 | 0.00 | 0.00 | 0.00 | 0.00 | 0.00 | 0.00 | 0.00 | 0.00 | 0.00 |
| Discriminant Analysis | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| Random Forest | Total | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| naive Bayes | Total | 0.13 | 0.13 | 0.13 | 0.13 | 0.13 | 0.13 | 0.13 | 0.13 | 0.13 | 0.13 |
| | RF_Heat | 0.33 | 0.33 | 0.33 | 0.33 | 0.33 | 0.33 | 0.33 | 0.33 | 0.33 | 0.33 |
| | Control | 0.00 | 0.00 | 0.00 | 0.00 | 0.00 | 0.00 | 0.00 | 0.00 | 0.00 | 0.00 |
| Neural Networks | Total | 0.00 | 0.00 | 0.00 | 0.00 | 0.13 | 0.13 | 0.13 | 0.00 | 0.00 | 0.00 |
| | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.20 | 0.20 | 0.20 | 0.00 | 0.00 |
| Support Vector Machines (SVM) | Total | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| Generalized Linear Models | Total | 0.36 | 0.25 | 0.50 | 0.60 | 0.13 | 0.38 | 0.00 | 0.00 | 0.00 | 0.00 |
| (GLM) | RF_Heat | 0.57 | 0.67 | 0.67 | 0.67 | 0.00 | 0.67 | 0.00 | 0.00 | 0.00 | 0.00 |
| | Control | 0.20 | 0.00 | 0.40 | 0.40 | 0.20 | 0.20 | 0.00 | 0.00 | 0.00 | 0.00 |

In the RF radiation-exposed group, 13 of the 40 genes were observed to increase in expression level while 27 were decreased in expression level (Table 6).

TABLE 6

| Affymetrix | | | | Expression in |
|---|---|---|---|---|
| Transcript | | | | RF exposed |
| cluster_id | NM | Gene | Gene full name | cells |
| 8068361 | NM_006933 | SLC5A3 | solute carrier family 5 | Up-regulated |
| 8131666 | NM_002214 | ITGB8 | integrin, beta 8 | Up-regulated |
| 7993807 | NM_020422 | TMEM159 | transmembrane protein 159 | Up-regulated |
| 7962559 | NM_018018 | SLC38A4 | solute carrier family 38, member 4 | Up-regulated |
| 8131069 | NM_001039966 | GPER | G protein-coupled estrogen receptor 1 | Up-regulated |
| 7909789 | NM_001135599 | TGFB2 | transforming growth factor, beta 2 | Up-regulated |
| 8059680 | NM_000867 | HTR2B | 5-hydroxytryptamine (serotonin) receptor 2B | Up-regulated |
| 8164200 | NM_012098 | ANGPTL2 | angiopoietin-like 2 | Up-regulated |
| 8126452 | NM_000287 | PEX6 | peroxisomal biogenesis factor 6 | Up-regulated |
| 8180061 | NM_000593 | TAP1 | transporter 1, ATP-binding cassette, sub-family B | Up-regulated |
| 7958019 | NM_018370 | DRAM | damage-regulated autophagy modulator | Up-regulated |
| 8025199 | NM_006702 | PNPLA6 | patatin-like phospholipase domain containing 6 | Up-regulated |
| 7950235 | NM_006645 | STARD10 | StAR-related lipid transfer (START) domain | Up-regulated |
| | | | containing 10 | |
| 7993776 | NM_030941 | LOC81691 | exonuclease NEF-sp | Down-reulated |
| 8067985 | NM_004540 | NCAM2 | neural cell adhesion molecule 2 | Down-reulated |
| 8097128 | NM_001034194 | EXOSC9 | exosome component 9 | Down-reulated |
| 8024238 | NM_001280 | CIRBP | cold inducible RNA binding protein | Down-reulated |
| 8019576 | BC063625 | KRTAP2-4 | keratin associated protein 2-4 | Down-reulated |
| 7989915 | NM_017858 | TIPIN | TIMELESS interacting protein | Down-reulated |
| 8129763 | NM_001099286 | FAM54A | family with sequence similarity 54, member A | Down-reulated |
| 8055426 | NM_005915 | MCM6 | minichromosome maintenance complex component 6 | Down-reulated |
| 8124430 | NM_005320 | HIST1H1D | histone cluster 1, H1d | Down-reulated |
| 7910997 | NM_130398 | EXO1 | exonuclease 1 | Down-reulated |
| 7916167 | NM_004153 | ORC1L | origin recognition complex, subunit 1-like (yeast) | Down-reulated |
| 7948904 | NR_002562 | SNORD28 | small nucleolar RNA, C/D box 28 | Down-reulated |
| 8133434 | NM_022170 | EIF4H | eukaryotic translation initiation factor 4H | Down-reulated |
| 8073062 | NM_004900 | APOBEC3B | apolipoprotein B mRNA editing enzyme, catalytic | Down-reulated |
| | | | polypeptide-like 3B | |
| 8117426 | NM_003524 | HIST1H2BH | histone cluster 1, H2bh | Down-reulated |
| 7926259 | NM_182751 | MCM10 | minichromosome maintenance complex component | Down-reulated |
| | | | 10 | |
| 7948902 | NR_002559 | SNORD29 | small nucleolar RNA, C/D box 29 | Down-reulated |
| 7948908 | NR_002564 | SNORD26 | small nucleolar RNA, C/D box 26 | Down-reulated |
| 8167234 | NM_006743 | RBM3 | RNA binding motif (RNP1, RRM) protein 3 | Down-reulated |
| 7948900 | NR_002561 | SNORD30 | small nucleolar RNA, C/D box 30 | Down-reulated |
| 8071212 | NM_003504 | CDC45L | CDC45 cell division cycle 45-like (S. cerevisiae) | Down-reulated |
| 8117594 | NM_003521 | HIST1H2BM | histone cluster 1, H2bm | Down-reulated |
| 7968563 | NM_002915 | RFC3 | replication factor C (activator 1) 3, 38 kDa | Down-reulated |
| 8124380 | NM_005325 | HIST1H1A | histone cluster 1, H1a | Down-reulated |
| 7971653 | NR_002612 | DLEU2 | deleted in lymphocytic leukemia 2 (non-protein | Down-reulated |
| | | | coding) | |
| 7948906 | NR_002563 | SNORD27 | small nucleolar RNA, C/D box 27 | Down-reulated |
| 7948910 | NR_002565 | SNORD25 | small nucleolar RNA, C/D box 25 | Down-reulated |

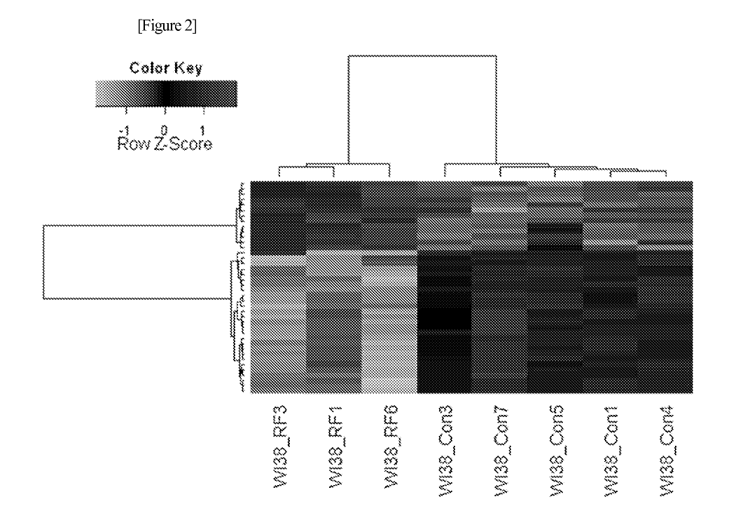

The relative expression levels of the 40 genes are depicted in the heat map of FIG. 2.

2-3. Prediction Using Genes Selected with Data of Training Sets

Eight samples divided sub-grouped to one test set and seven training sets and a t-test was conducted with the sets. The genes were selected in the increasing order of p value and applied to prediction algorithms to predict RF radiation exposure. The results are summarized in Table 7, below. Pre-validation indicated that Diagonal Linear Discriminant Analysis, and support vector machine were the most effective (prediction accuracy 100%).

TABLE 7

| Algorithm of supervised | Prediction | Number of selected features (genes) by moderated t test | | | | | | | | | | |
|---|---|---|---|---|---|---|---|---|---|---|---|---|
| machine learning | error rate | 5 | 10 | 15 | 20 | 25 | 30 | 35 | 40 | 45 | 50 | 75 |
| k-Nearest Neighbour | Total | 0.13 | 0.00 | 0.13 | 0.00 | 0.13 | 0.13 | 0.13 | 0.13 | 0.13 | 0.13 | 0.13 |
| | RF_Heat | 0.33 | 0.00 | 0.33 | 0.00 | 0.33 | 0.33 | 0.33 | 0.33 | 0.33 | 0.33 | 0.33 |
| | Control | 0.00 | 0.00 | 0.00 | 0.00 | 0.00 | 0.00 | 0.00 | 0.00 | 0.00 | 0.00 | 0.00 |
| Linear Discriminant | Total | 0.25 | 0.00 | 0.00 | 0.00 | 0.00 | 0.13 | 0.13 | 0.13 | 0.00 | 0.00 | 0.00 |
| Analysis (LDA) | RF_Heat | 0.33 | 0.00 | 0.00 | 0.00 | 0.00 | 0.33 | 0.33 | 0.33 | 0.00 | 0.00 | 0.00 |
| | Control | 0.20 | 0.00 | 0.00 | 0.00 | 0.00 | 0.00 | 0.00 | 0.00 | 0.00 | 0.00 | 0.00 |
| Diagonal Linear | Total | 0.00 | 0.00 | 0.00 | 0.00 | 0.00 | 0.00 | 0.00 | 0.00 | 0.00 | 0.00 | 0.00 |
| Discriminant Analysis | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Random Forest | Total | 0.00 | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| naive Bayes | Total | 0.25 | 0.13 | 0.13 | 0.25 | 0.25 | 0.25 | 0.25 | 0.25 | 0.25 | 0.25 | 0.25 |
| | RF_Heat | 0.33 | 0.33 | 0.33 | 0.67 | 0.67 | 0.67 | 0.67 | 0.67 | 0.67 | 0.67 | 0.67 |
| | Control | 0.20 | 0.00 | 0.00 | 0.00 | 0.00 | 0.00 | 0.00 | 0.00 | 0.00 | 0.00 | 0.00 |
| Neural Networks | Total | 0.00 | 0.00 | 0.00 | 0.00 | 0.13 | 0.13 | 0.13 | 0.13 | 0.13 | 0.13 | 0.13 |
| | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.20 | 0.20 | 0.20 | 0.20 | 0.20 | 0.20 | 0.20 |
| Support Vector Machines | Total | 0.00 | 0.00 | 0.00 | 0.00 | 0.00 | 0.00 | 0.00 | 0.00 | 0.00 | 0.00 | 0.00 |
| (SVM) | RF_Heat | 0.00 | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 | 0.00 |
| Generalized Linear Models | Total | 0.25 | 0.38 | 0.25 | 0.13 | 0.13 | 0.13 | 0.13 | 0.38 | 0.38 | 0.38 | 0.63 |
| (GLM) | RF_Heat | 0.33 | 0.33 | 0.33 | 0.00 | 0.00 | 0.33 | 0.33 | 0.67 | 0.33 | 0.33 | 0.67 |
| | Control | 0.20 | 0.40 | 0.20 | 0.20 | 0.20 | 0.00 | 0.00 | 0.20 | 0.40 | 0.40 | 0.50 |

| Algorithm of supervised | Prediction | Number of selected features (genes) by moderated t test | | | | | | | | | |
|---|---|---|---|---|---|---|---|---|---|---|---|
| machine learning | error rate | 100 | 125 | 150 | 175 | 200 | 225 | 250 | 275 | 300 | 325 |
| k-Nearest Neighbour | Total | 0.13 | 0.13 | 0.13 | 0.13 | 0.13 | 0.13 | 0.25 | 0.25 | 0.25 | 0.25 |
| | RF_Heat | 0.33 | 0.33 | 0.33 | 0.33 | 0.33 | 0.33 | 0.67 | 0.67 | 0.67 | 0.67 |
| | Control | 0.00 | 0.00 | 0.00 | 0.00 | 0.00 | 0.00 | 0.00 | 0.00 | 0.00 | 0.00 |
| Linear Discriminant | Total | 0.00 | 0.00 | 0.00 | 0.00 | 0.00 | 0.00 | 0.00 | 0.00 | 0.00 | 0.00 |
| Analysis (LDA) | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| Diagonal Linear | Total | 0.00 | 0.00 | 0.00 | 0.00 | 0.00 | 0.00 | 0.00 | 0.00 | 0.00 | 0.00 |
| Discriminant Analysis | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| Random Forest | Total | 0.00 | 0.13 | 0.00 | 0.00 | 0.00 | 0.00 | 0.00 | 0.13 | 0.13 | 0.13 |
| | RF_Heat | 0.00 | 0.33 | 0.00 | 0.00 | 0.00 | 0.00 | 0.00 | 0.33 | 0.33 | 0.33 |
| | Control | 0.00 | 0.00 | 0.00 | 0.00 | 0.00 | 0.00 | 0.00 | 0.00 | 0.00 | 0.00 |
| naive Bayes | Total | 0.25 | 0.25 | 0.25 | 0.13 | 0.13 | 0.13 | 0.25 | 0.25 | 0.25 | 0.25 |
| | RF_Heat | 0.67 | 0.67 | 0.67 | 0.33 | 0.33 | 0.33 | 0.67 | 0.67 | 0.67 | 0.67 |
| | Control | 0.00 | 0.00 | 0.00 | 0.00 | 0.00 | 0.00 | 0.00 | 0.00 | 0.00 | 0.00 |
| Neural Networks | Total | 0.13 | 0.13 | 0.13 | 0.13 | 0.13 | 0.13 | 0.13 | 0.13 | 0.13 | 0.13 |
| | RF_Heat | 0.00 | 0.00 | 0.00 | 0.00 | 0.00 | 0.00 | 0.00 | 0.00 | 0.00 | 0.00 |
| | Control | 0.20 | 0.20 | 0.20 | 0.20 | 0.20 | 0.20 | 0.20 | 0.20 | 0.20 | 0.20 |
| Support Vector Machines (SVM) | Total | 0.00 | 0.00 | 0.00 | 0.00 | 0.00 | 0.00 | 0.00 | 0.00 | 0.00 | 0.00 |
| | RF_Heat | 0.00 | 0.00 | 0.00 | 0.00 | 0.00 | 0.00 | 0.00 | 0.00 | 0.00 | 0.00 |
| | Control | 0.00 | 0.00 | 0.00 | 0.00 | 0.00 | 0.00 | 0.00 | 0.00 | 0.00 | 0.00 |
| Generalized Linear Models | Total | 0.38 | 0.13 | 0.13 | 0.25 | 0.13 | 0.25 | 0.25 | 0.25 | 0.25 | 0.38 |
| (GLM) | RF_Heat | 0.33 | 0.00 | 0.33 | 0.33 | 0.00 | 0.33 | 0.67 | 0.67 | 0.67 | 0.67 |
| | Control | 0.40 | 0.20 | 0.00 | 0.20 | 0.20 | 0.20 | 0.00 | 0.00 | 0.00 | 0.20 |

Therefore, the 40 genes that change in expression level with the most significance are useful as biomarkers and the analysis thereof with the algorithms Diagonal Linear Discriminant Analysis or Support Vector Machines allows the accurate prediction of the exposure of cells or a subject of interest to electromagnetic radiation.

As described above, the present invention provides compositions, kits and methods for diagnosis of exposure to electromagnetic radiation (e.g., an agent capable of measuring at an mRNA or protein level the expression level of genes given in Table 6 and methods of using the agent).

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A composition for diagnosis of exposure to electromagnetic radiation, comprising an agent capable of measuring at an mRNA or protein level the expression level of genes given in Table 6.

2. The composition according to claim 1, wherein the electromagnetic radiation has a frequency of 1762.5 MHz and an intensity of 60 W/kg or higher SAR (specific absorption rate).

3. The composition according to claim 1, wherein the agent capable of measuring at an mRNA level the expression level of the genes comprises pairs of primers or probes binding specifically to the genes.

4. The composition according to claim 1, wherein the agent capable of measuring at a protein level the expression level of the genes comprises an antibody specific for the proteins encoded by the genes.

5. A kit for diagnosis of exposure to electromagnetic radiation, comprising the composition of claim 1.

6. The kit according to claim 5, being in a form of an RT-PCR kit, a microarray chip kit, or a protein chip kit.

7. The kit according to claim 6, wherein the microarray chip kit comprises the genes of Table 6, or their oligonucleotide segments or complementary strand molecules which are clustered on a substrate.

8. A method for detecting diagnostic marker genes of Table 6, comprising:
measuring at an mRNA or protein level the expression level of genes given in Table 6 in a sample from a subject; and
comparing the expression level of the genes with that of corresponding genes from a normal control.

9. A method for diagnosis of exposure to electromagnetic radiation, comprising:
measuring at an mRNA or protein level the expression level of genes given in Table 6 in a sample from a subject; and
comparing the expression level of the genes with that of corresponding genes from a normal control.

10. The method according to claim 9, wherein the genes of GenBank Nos. NM_{—}006933, NM_{—}002214, NM_{—}020422, NM_{—}018018, NM_{—}001039966, NM_{—}001135599, NM_{—}000867, NM_{—}012098, NM_{—}000287, NM_{—}000593, NM_{—}018370, NM_{—}006702, and NM_{—}006645 selected from among those given in Table 6 are increased in mRNA or protein expression level upon exposure of the subject to electromagnetic radiation.

11. The method according to claim 9, wherein the genes of GenBank Nos. NM_{—}030941, NM_{—}004540, NM_{—}001034194, NM_{—}001280, BC063625, NM_{—}017858, NM_{—}001099286, NM_{—}005915, NM_{—}005320, NM_{—}130398, NM_{—}004153, NR_{—}002562, NM_{—}022170, NM_{—}004900, NM_{—}003524, NM_{—}182751, NR_{—}002559, NR_{—}002564, NM_{—}006743, NR_{—}002561, NM_{—}003504, NM_{—}003521, NM_{—}002915, NM_{—}005325, NR_{—}002612, NR_{—}002563, and NR_{—}002565 selected from among those given in Table 6 are decreased in mRNA or protein expression level upon exposure of the subject to electromagnetic radiation.

12. The method according to claim 9, wherein the expression of the genes is measured at an mRNA level using pairs of primers or probes binding specifically to the genes.

13. The method according to claim 9, wherein the expression of the genes is measured at an mRNA level using a method selected from among RT-PCR, competitive RT-PCR, real-time RT-PCR, RNase protection assay, Northern blotting, DNA microarray chip and a combination thereof.

14. The method according to claim 13, wherein the measurement using DNA microarray chip comprises:
isolating mRNAs of the marker genes given in Table 6 from samples from both a subject and a normal control;
synthesizing cDNAs from the mRNAs, with respective fluorescent material incorporated thereinto;
hybridizing the fluorescent-labeled cDNAs with a DNA microarray chip; and
analyzing the hybridized DNA microarray chip to compare mRNA expression levels of the marker genes given in Table 6 between the subject and the normal control.

15. The method according to claim 9, wherein the expression of the genes is measured at a protein level using a method selected from among Western blotting, ELISA, radioimmunoassay, radioimmunodiffusion, Ouchterlony immunodiffusion, rocket immunoelectrophoresis, histoimmunostaining, immunoprecipitation assay, complement fixation assay, FACS, protein chip and a combination thereof.

16. The method according to claim 9, wherein the sample from the subject is fibroblast WI-38 cells.
